(19) United States
(12) Patent Application Publication
(10) Pub. No.: US 2005/0203186 A1
(43) Pub. Date: Sep. 15, 2005

(54) Medicaments containing active ingredients which lower the level of cholesterol with time-delayed active ingredient release

(76) Inventor: Peter Kraass, Munchen (DE)

Correspondence Address: Stephen M Haracz;Bryan Cave, 1290 Avenue of the Americas, New York, NY, 10104-3300, US

(21) Appl. No.: 10/506,583

(22) PCT Filed: Mar. 6, 2003
(86) PCT No.: PCT/EP03/02328

§ 371 Date: Apr. 18, 2005

(30) Foreign Application Priority Data
Mar. 7, 2002 (DE) 102 09 979.0

(51) Int. Cl. A61K 31/194; A61K 9/22

(52) U.S. Cl. 514/571; 424/468

(57) ABSTRACT

The invention relates to a medicament containing at least one active ingredient which lowers the cholesterol in the blood, characterized in that it has means for providing release characteristics for the active ingredient with which the active ingredient is released with at least two different release rates, specifically with a first release rate in a first period and with a second release rate, which is higher than the first release rate, in a subsequent second period, where the second period starts 2 to 12 hours after administration of the medicament.

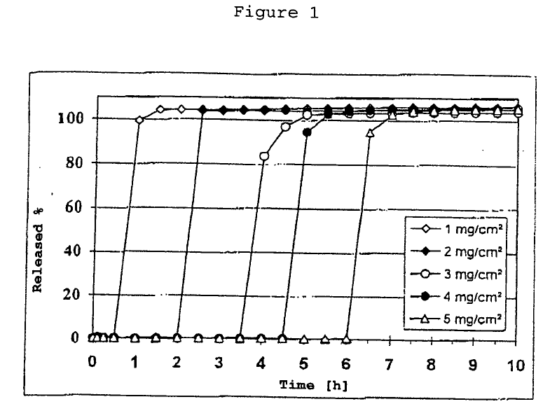

The invention relates to medicaments comprising one or more active ingredients which lowers (lower) the cholesterol level in the blood, in particular one or more HMG-CoA reductase inhibitors, or one or more drugs from the class of statins. Other suitable active ingredients are, for example, fibrates. The medicaments of the invention release the active ingredient in a time-delayed fashion, meaning that there is initially no or only small release over a period and, after this period, release of the active ingredient as fast as possible.

Drugs which lower the cholesterol level in the blood, and medicaments containing them, are known in the art. In particular, HMG-CoA reductase inhibitors reduce the plasma cholesterol level through inhibition of cholesterol biosynthesis, which takes place mainly in the liver, and are therefore employed in patients with elevated cholesterol level. However, HMG-CoA reductase inhibitors, which include drugs such as fluvastatin, simvastatin, atorvastatin, pravastatin, cerivastatin, lovastatin, nisvastatin, dolvastatin, bervastatin and rosuvastatin, and further statins, are not without side effects and are not well tolerated by all patients. This also applies to other drugs which lower the cholesterol level in the blood. Marked side effects, which are described in detail in the relevant literature, may occur, in particular with higher dosages leading to high peak plasma levels of the active ingredient.

To reduce the side effect, controlled release forms have been proposed in the prior art. For example, EP-A 465 096 discloses medicaments with uniform sustained release of the HMG-CoA reductase inhibitor, meaning that active ingredient release starts as directly as possible after administration of the medicament at an essentially constant (“sustained release”) or slightly decreasing (“prolonged release”) rate over a period of several hours. This leads to a substantially constant active ingredient level, which is not too high, being set up in the patient over a prolonged period. It is possible in this way to extend the activity and duration of action and thus lower the cholesterol concentration to a greater degree.

Similar formulations are disclosed in WO 00/21525.

However, it has emerged in practice that these medicaments as disclosed in principle in WO 00/21525 or EP-A 465 096 are not yet completely satisfactory. Thus, the extent of cholesterol production in the human body varies over the course of the day, with the result that with controlled release forms like those disclosed in WO 00/21525 and EP-A 465 096, higher blood levels would be more effective at some times (when there is elevated cholesterol production), and a blood level concentration which is in principle still unnecessarily high is present at other times (when there is virtually no cholesterol production). In order to achieve a maximally effective reduction of the cholesterol level in the blood, the release even with a controlled release drug form would have to be sufficiently high for it to be effective when cholesterol production is elevated, but this again may lead to considerable side effects.

With the normally employed medicaments with an HMG-CoA reductase inhibitor as active ingredient, there is rapid release of active ingredient from the drug form and a rapid rise in level at the site of action and, depending on the elimination half-life of the particular substance, it is also eliminated more or less quickly from the body and therefore the concentration available at the site of action is possibly no longer sufficiently high at the time when the principal biosynthesis of cholesterol takes place. This phenomenon is particularly pronounced with fast-release drug forms of HMG-CoA reductase inhibitors with a very short elimination half-life, such as fluvastatin. Although the problem is partly solved by the controlled release drug forms described in EP-A 465 096 and WO 00/21525, in these cases a sometimes considerable proportion of the dose is released long before the actually desired effect and thus enters the body. The drug is able to show only a small effect during this time, and can cause side effects to a greater extent. At the time of the actually desired effect, part of the dose has already been consumed, and the concentration of the active ingredient necessary for inhibition of cholesterol biosynthesis is thus only low. This could be counteracted at the most by a higher dose, but this is undesirable for the stated reasons. Alternatively, an HMG-CoA reductase inhibitor with a long elimination half-life could be employed, such as atorvastatin. Substances of this type circulate in the blood long enough to achieve a large effect even long after the administration. However, the disadvantage of substances of this type is that continuously high and effective blood levels are reached, leading to an increased burden on the body and increasing the extent of side effects.

It is an object of the invention to provide a medicament with which the cholesterol level can be reduced in patients, with a reduction in the side effects which occur with medicaments known in the art, and with which the active ingredient can be administered in a reduced dose without the efficacy of the composition suffering therefrom.

This object is achieved according to the invention by proposing a medicament containing at least one active ingredient which lowers the cholesterol level in the blood, in particular an HMG-CoA reductase inhibitor, which has means for providing release characteristics for the active ingredient with which the active ingredient is released with at least two different release rates, specifically with a first release rate in a first period and with a second release rate, which is higher than the first release rate, in a subsequent second period, where the second period starts 2 to 12 hours after administration of the medicament.

The medicaments of the invention preferably display delayed release.

The medicament of the invention takes account of the fact that cholesterol production in the human body takes place mainly in the second half of the night and therefore inhibition of the synthesis ought not to take place until some time after administration of the medicament (usually in the evening). With the medicament of the invention, only very little or preferably no active ingredient is released initially over a first adjustable period, taking account of the fact that there is only a small extent of cholesterol biosynthesis in the first half of the night. The level of drug is instead allowed to increase to a level in the blood which is as high as possible as close as possible to the time when cholesterol synthesis principally takes place, and thus the efficacy of the active ingredient is increased with the same dose compared with known pharmaceutical forms. In addition, the same effect can also be achieved at the necessary time without the need for high initial doses or long-lasting high blood levels.

The medicaments of the invention thus have the advantage that immediately after administration, which normally takes place some hours before cholesterol biosynthesis reaches its peak in the second half of the night, no active ingredient or only little active ingredient enters the bloodstream and thus active ingredient is not consumed and no side effects can be caused either. Only after a lag time, which is chosen so that it extends into the second half of the night, is the active ingredient released as fast as possible and thus reaches the actual site of action in a very high dose and is there able to inhibit the cholesterol biosynthesis which is taking place to an increased extent. This has the advantage that the body is not burdened with active ingredient before the actual cholesterol biosynthesis needs to be inhibited and, at the same time, the total active ingredient of the medicament is available when the inhibition is necessary.

Means and methods for achieving delayed release of active ingredient are known. Some means preferred according to the invention are described by way of example below.

A certain delayed release is achieved for example by providing the medicament with a coating which is insoluble in the acidic medium of the stomach and which dissolves in the intestine when the pH rises. The term used is pH control, and corresponding drug forms are in wide use as enteric-coated medicaments. However, pH control on its own is unsuitable to achieve the delayed release desired according to the invention, because it is exceptionally inaccurate. The residence time of an enteric-coated medicament in the stomach depends on many conditions which cannot be fixed, especially the time, the nature and the amount of food intake, and the size and density of the drug form. A pH-controlled coating could at the most achieve the desired release characteristics very inaccurately and with considerable restrictions concerning the time of administration. In addition, the lag times which can be attained by pH control are usually too short for the objective desired in this case. The control of release with the medicaments therefore does not take place according to the invention through a pH-controlled coating or an enteric coating. Although the medicaments of the invention may include an enteric coating in order to ensure resistance to gastric juice, by which means the lag time can be further extended, even if they have an enteric coating they additionally have further means to achieve the desired release characteristics.

Suitable means and methods for producing medicaments with delayed release are described for example in EP-A 210 540, the disclosure of which is incorporated herein by reference. Medicaments based on the technology of EP-A 210 540 are preferred according to the invention. The medicaments described in EP-A 210 540 are so-called time-controlled explosion systems which comprise the drug together with a swelling agent and which are enveloped by a water-insoluble, non-pH-dependent coating. In these systems, the active ingredient is released at a higher rate after a defined lag time.

The medicaments described therein consist for example of granules comprising the active ingredient and one or more swelling agents, and conventional excipients and additives, or of pellets which are provided with a coating, which contain the active ingredient and the swelling agent and, where appropriate, conventional additives. EP-A 210 540 likewise discloses tablets which are likewise preferred according to the invention. It is also possible for swelling agent and active ingredient to be present separately, for example in different layers of a pellet or of a tablet. It is essential that the active ingredient and the swelling agent are enveloped by a water-insoluble layer which is, however, not completely impermeable to water. Administration is followed by water uptake in the gastrointestinal tract, eventually resulting in the swelling of the swelling substances being so great that the water-insoluble layer bursts. The active ingredient is then immediately released.

The advantage of these systems is that the release is virtually uninfluenced by the solubility or the rate of dissolution of the active ingredient or by the pH of the liquid in the gastrointestinal tract, and the delay until the active ingredient is released is therefore independent of the residence time of the medicament in the stomach.

It is possible by mixing explosion systems of this type with a plurality of different time delays to provide medicaments which release pulses of active ingredients at different times. This may in the present case for example be advantageous in order to provide pulses of an HMG-CoA reductase inhibitor at different times at which cholesterol biosynthesis increases. It is likewise possible, for example, after a first lag time, for a first active ingredient which has a very short elimination half-life, such as fluvastatin, to be released rapidly in order to inhibit the cholesterol biosynthesis which occurs particularly extensively in the second half of the night and, after a further lag time, for a lower dose of an HMG-CoA reductase inhibitor, such as atorvastatin, which has a very long elimination half-life and which subsequently inhibits over a longer period the cholesterol biosynthesis which is taking place to a reduced extent, to be released.

EP-A 210 540 likewise describes explosion systems which have a plurality of active ingredient layers and a plurality of swelling agent layers and which therefore provide different release patterns. This disclosure and corresponding explosion systems of EP-A 210 540 are also relevant to the present invention and are included herein by reference.

The lag time for active ingredient release can be controlled for example via the thickness of the outer water-insoluble coating or the nature of the specific water-insoluble coating. Examples thereof are indicated in EP-A 210 540, to which reference is made in this regard. It is further possible for so-called pore formers, which dissolve in water and thus make possible or speed up access of water to the swelling agent, to be present in the water-insoluble coating. With an embodiment of this type it is possible to adjust the lag time also via the amount of pore former.

The swelling agent preferred according to the invention is a customary disintegrant used for tablets, such as, for example, crosslinked sodium carboxymethylstarch, low-substituted sodium carboxymethylstarch, crosslinked sodium carboxymethylcellulose, crosslinked polyvinylpyrrolidone, low-substituted hydroxypropylcellulose, starch, highly swellable ion exchange resins such as Amberlite® or cholestyramine, or similar swelling agents.

The water-insoluble films preferably consist of customary pharmaceutical film polymers such as, for example, ethylcellulose, cellulose acetate, polyinyl acetate, acrylates, and mixtures of these polymers in combination with customary excipients such as plasticizers, pigments, non-stick substances, dispersing aids, buffer substances, fillers and pore formers.

Preferred water-insoluble polymers, swelling agents or disintegrants and suitable excipients are likewise indicated in EP-A 210 540, to which reference is made in this regard. Instead of the active ingredients mentioned in EP-A 210 540, the active ingredients used according to the invention are those which lower the cholesterol level, in particular HMG-CoA reductase inhibitors.

It is preferred according to the invention for an intermediate layer, which is preferably water-soluble, to be present between the water-insoluble layer and the active ingredient-containing core. It is possible by an intermediate layer of this type for example to round off the band edges resulting during the tableting, and through use of appropriate swelling agents to increase further the expansion in volume of the drug form on contact with water. Such an intermediate layer can consist of a water-soluble polymer such as, for example, hydroxypropylmethylcellulose, hydroxypropylcellulose, polyvinylpyrrolidone, polyvinylpyrrolidone/polyvinyl acetate copolymers or further water-soluble polymers customarily used in pharmacy, and further customary excipients such as non-stick substances, plasticizers, pigments and fillers. The water-soluble intermediate layer may likewise have the aforementioned swelling agents. It is possible according to the invention for the swelling agents to be present either in the active ingredient-containing layer (or the active ingredient-containing core) or in the water-soluble intermediate layer or both in the active ingredient-containing layer and in the intermediate layer.

It is particularly preferred according to the invention for the medicament to be in the form of particles which are preferably microtablets or pellets. The particles may have for example a size of for example from 1 to 4 mm, and a plurality of particles are normally combined in a customary hard or soft gelatin capsule. A capsule containing a single dose comprises the dose distributed on a particular number of particles (e.g. microtablets or pellets), the number being determined by the size of the capsule, the size of the contained particles (microtablets or pellets), the active ingredient loading etc.

Reference is made hereinafter exclusively to microtablets and pellets, but the statements apply correspondingly also to other particles.

It is likewise possible according to the invention to compress the microtablets or, for example, pellets to a tablet, in which case, however, care must be taken that the coatings of the microtablets or pellets which influence the delayed release are not damaged.

It is additionally possible for an amount of microtablets or pellets corresponding to the desired dosage to be provided in sachets (possibly mixed with additional excipients such as fillers and flavorings), glass bottles or similar primary packagings.

The proportion of swelling agent in the medicaments of the invention depends on the desired lag time and is generally from 5 to 90%, preferably 10 to 80%, more preferably 10 to 60%, even more preferably 10 to 40%, based on the weight of the swelling agent-containing core or the swelling agent-containing layer.

Likewise preferred according to the invention are systems for “pulsatile” release of active ingredients, like those described for example in U.S. Pat. No. 5,229,131, to which reference is made in this regard. Preferred according to the invention are the medicaments described in U.S. Pat. No. 5,229,131 which comprise two different subunits. The individual subunits comprise a core which is provided with a coating, the two subunits differing through the nature of the coating. The coating comprises water-permeable polymers which are, however, impermeable to the active ingredient and have a particular tensile strength and a particular maximum elongation, resulting in the polymer shell being destroyed after a predetermined time in the aqueous medium of the gastrointestinal tract and releasing the active ingredient-containing core. Pulsatile administration of active ingredients is possible through the choice of different polymers. Concerning the production of corresponding systems, reference is made to U.S. Pat. No. 5,229,131 in its entirety.

A process for producing solid drug forms which can be used orally and have controlled active ingredient delivery is also disclosed in WO 98/48782, although the delayed release of the drug forms mentioned therein is caused solely by an enteric coating.

It is possible and preferred for the medicaments of the invention also to be produced in a manner like that described in detail in WO 98/51287, to the disclosure of which in this regard reference is likewise made.

Medicaments of the invention can likewise preferably be produced in a manner like that described in WO 99/51209, to which reference is likewise made in this regard. The medicaments described therein show prolonged release over a first period before a very rapid pulsatile release takes place in a second period.

Medicament formulations based on the principle of time-controlled explosion systems are likewise disclosed in DE-A 199 23 817, to the disclosure of which, especially also concerning the production of such medicaments, express reference is likewise made.

The publications detailed above disclose by way of example various methods for bringing about the delayed release as is intended according to the invention (although according to the invention delayed release is not to take place exclusively through an enteric coating). The list is meant to be only way of example, not definitive, and it is possible in principle to use any process for obtaining a medicament of the invention with delayed release.

The release of the active ingredient used takes place according to the invention with an increased rate (“faster release” or “fast release”) after a delay of from 2 to 12 hours. The lag time is preferably at least 3 hours, more preferably at least 4 hours. The lag time is preferably not more than 10 hours, more preferably not more than 8 hours, most preferably not more than 6 hours. During the lag time there is “slow release”, by which is meant that preferably there is no release of the active ingredient, so that the “slow” release rate is 0% of active ingredient per 10-minute interval. However, it is also possible for a small amount of active ingredient to be released even during the lag time. In this case, the release rate in the lag period (“slow release”) is preferably not more than 5%, more preferably not more than 2%, of the active ingredient, preferably of the HMG-CoA reductase inhibitor, per 10-minute interval. In total, preferably not more than 30% by weight, more preferably not more than 20% by weight and even more preferably not more than 10% by weight, of the active ingredient is released during the lag interval.

After the end of the lag time, the active ingredient is released at a considerably greater rate (“fast release”), it being preferred according to the invention for the release rate after the lag time to be as high as possible. It is preferably at least 6%, more preferably at least 10%, even more preferably 15% or more, in particular immediate or at least 20% of active ingredient per 10-minute interval. If release of the active ingredient takes place even during the lag interval at a lower rate, according to the invention the release rate after the lag period is preferably at least twice as high as the release rate during the lag period, more preferably it is at least three times as high, and even more preferably at least four times as high.

The period in which fast release takes place is preferably not more than 5 hours, more preferably not more than 3 hours and even more preferably not more than 2 hours, in particular not more than 1 hour. In the most preferred embodiment, release of the active ingredient takes place explosively through bursting of a coating which previously prevented release.

The release rate during the period of fast or “faster” release is higher than during the period of slow (or “slower”) release.

It is preferred according to the invention for the release of active ingredient at the end of the period of fast release to be complete, but at least 50%, more preferably 80%, even more preferably 90% or more.

Likewise preferred according to the invention is an embodiment in which controlled release takes place after a lag interval. In this case, the period of fast release (in this case controlled release) is preferably 3 to 9 hours, in particular 3 to 6 hours.

If the medicament of the invention is a system in which the active ingredient is released in more than one pulse, meaning that there is a plurality of lag periods, the first lag period is preferably in a range from 2 to 10 hours, more preferably from 3 to 6 hours, and the second lag period (starting after the first lag period) is in a range from 1.5 to 5 hours, more preferably in a range from 2 to 4 hours. With this embodiment, the ranges preferred for the release rate of each pulse are the same as described above for the embodiment with which release takes place in one pass. With these embodiments of the invention in which the release takes place in more than one, preferably in two, pulses after appropriately two lag periods, the active ingredient released in both pulses can be the same. This is preferred for example when it is intended to inhibit several peaks of cholesterol biosynthesis by a single administration of the medicament. However, it is also possible for a different active ingredient to be released in each pulse, for example in a first pulse an HMG-CoA reductase inhibitor which, like fluvastatin, has a low elimination half-life, and in a second pulse an HMG-CoA reductase inhibitor, such as atorvastatin, which has a long elimination half-life and which is preferably released only when the HMG-CoA reductase inhibitor with a short elimination half-life has been essentially removed from the body, and which then sets up a substantially constant active ingredient level. However, it is also possible to release the HMG-CoA reductase inhibitor with the long elimination half-life in a first pulse, which then ensures a basic level of HMG-CoA reductase inhibitor in the blood, onto which an HMG-CoA reductase inhibitor with a short elimination half-life is released in a second pulse and is then intended to cope with individual peaks of cholesterol biosynthesis. In this case, the first lag period is shorter than the second lag period.

It is likewise possible according to the invention to administer in a first pulse, for example, an HMG-CoA reductase inhibitor, and in a second pulse another active ingredient, e.g. an active ingredient from the class of fibrates or, preferably, to administer in a first pulse an active ingredient from the class of fibrates and in a second pulse an HMG-CoA reductase inhibitor.

The active ingredients of the invention are active ingredients which lower the cholesterol level in the blood, with preference for an active ingredient from the class of fibrates. However, customary HMG-CoA reductase inhibitors are particularly preferred, some of which are described for example in EP-A 465 096.

The HMG-CoA reductase inhibitors which are to be administered with the medicaments of the invention are preferably statins, in particular fluvastatin, simvastatin, atorvastatin, pravastatin, cerivastatin, nisvastatin, dolvastatin, bervastatin, rosuvastatin and lovastatin, their enantiomers or enantiomer mixtures, and pharmaceutically acceptable salts, solvates and hydrates of these compounds. Particular preference is given according to the invention to fluvastatin, which has a very low elimination half-life and therefore with which the advantage described according to the invention is particularly evident. Correspondingly, cerivastatin, which likewise has a low elimination half-life, is also preferred.

Preference is given according to the invention to a medicament for oral administration which is either in tablet form or in the form of a customary capsule or with which the individual units are provided in suitable primary packagings permitting individual dosage.

The medicament may consist for example of a plurality of pellets or microtablets which are then compressed to a tablet, or are packed into a soft or hard gelatin capsule. If release takes place via an explosion system, it is possible for the individual pellets or microtablets to be covered for example by a water-insoluble layer and to contain a swelling agent, or else for the finished capsule or the compressed tablet to be covered by a water-insoluble layer of this type. In this case, the swellable substance can be present either in each individual microtablet or each individual pellet (in the core and/or a covering), or in a covering of the tablet or the capsule. It is, of course, also possible to choose a monolithic drug form, e.g. a unitary tablet, which in the case of an explosion system includes a water-insoluble but water-permeable layer beneath which the active ingredient and the swelling substance, and further customary excipients and additives, as explained above, are disposed either in a mixture or in different layers.

Besides those described above, there is also a number of other procedures for achieving delayed release. The principal procedures for attaining delayed release which can be used to produce the medicaments of the invention are summarized below.

1. Splitting of a water-insoluble film because of the expansion in volume of the core, the expansion in volume being achieved through the swelling of a highly swellable excipient on uptake of water, as described in detail above. This procedure is preferred according to the invention.

2. Splitting of a semipermeable film because of an expansion in the volume of the core, the expansion in volume being achieved by osmotic penetration of water into the core and subsequent expansion of the drug form (this system is described for example in Schultz, Kleinebudde, J. Contr. Rel. 47 (1997), pp. 181-189 and pp. 191-199).

3. A further possibility is to apply an erodable outer layer, which slowly erodes in the gastrointestinal tract and releases the active ingredient only after erosion is complete, to a drug-containing drug form. The erosion can be achieved either by aqueous dissolution or by enzymatic degradation. This procedure is described for example in Matsuo et al., Int. J. Pharm. 138 (1996), pages 225-235 and in Gazzaniga et al., Eur. J. Pharm. Biopharm. 40 (1994), pages 246-250, and is likewise preferred according to the invention.

4. Likewise possible according to the invention is the time-controlled opening of a closure of an insoluble capsule. For this purpose, a customary drug capsule can be closed with a special closure system which undergoes time-controlled expulsion through swelling or expansion in the volume of the contents of the capsule after a particular time, or else undergoes time-controlled erosion or decomposition by enzymes in the digestive tract. Systems of this type are described for example in Binns et al., Proceed. Intern. Symp. Control. Rel. Bioact. Metr. 21 (1994), pages 260-261.

5. Likewise possible are systems in which the coating and/or a tablet mantle in the case of mantle tablets are enzymatically degraded in the digestive tract.

6. Finally, delayed release can also be achieved by providing a coating which contains an incompatible excipient, the incompatibility occurring only on entry of water into the drug form. It is possible for example to choose a coating of Eudragit RS which contains as excipient an organic acid such as succinic acid which slowly decomposes the coating after entry of water in the digestive tract. Systems of this type are described for example in Narisawa et al., Pharm. Res. 11 (1994), pages 111-116.

The lag time or dissolution rates are determined as described for example in the European Pharmacopoeia in Section 2.9.3 “Release of active ingredients from solid dosage forms” (EP 1997). Preference is given to a release bath normally known as paddle apparatus, or else so-called basket systems or even systems known as flow cell. Comparable systems are moreover described in the US Pharmacopeia.

The proportion of active ingredient released after particular time intervals from the tested drug form can be determined either by taking samples and subsequently analyzing them (e.g. UV-Vis or HPLC) or by using so-called on-line systems. In the case of the latter, the analytical method used to determine the drug concentration is integrated in the release system.

For the purposes of this specification the release is determined in accordance with the stipulations of the European Pharmacopoeia in Section 2.9.3 “Release of active ingredients from solid dosage forms” (EP 1997) the pH being 7.4 and the stirring speed being 50 rpm.

The medicaments of the invention can be particularly preferably produced as follows.

1. Tablets

| | |
|---|---|
| Active ingredient (preferably fluvastatin) | 33.3% |
| Croscarmellose | 10% |
| Lactose | 17.7% |
| Microcrystalline cellulose | 40% |
| Hydroxypropylmethylcellulose | 2% |
| Magnesium stearate | 0.5% |
| Colloidal SiO₂ | 0.5% |

Granules are prepared from the active ingredient, the lactose, the microcrystalline cellulose and the hydroxypropylmethylcellulose by wet granulation. These granules are dried and then mixed with croscarmellose, magnesium stearate and colloidal silica and compressed to tablets with, for example, a diameter of 2 mm and a mass of, for example, 6 mg.

2. Subcoat

The tablets from 1 are coated in a conventional way with a subcoat which has for example the following composition:

| | |
|---|---|
| Hydroxypropylcellulose | 45% |
| Titanium dioxide | 6% |
| Talc | 6% |
| PEG 6000 | 3% |
| Microcrystalline cellulose | 40% |

Approx. 2-5 mg/cm² (stated as pure polymer HPMC) are applied.

Alternatively, it is also preferred for a subcoat to be applied by the powder layering technology, in which case a polymer which swells greatly on contact with water (see above) is applied as powder (possibly with the addition of appropriate excipients) to microtablets which are simultaneously moistened with an aqueous binder solution (e.g. HMPC, povidone, HPC or others), with the aim of rounding off the band edges and simultaneously applying a substance which swells greatly.

3. Water-Insoluble Film

The tablets from 2 are coated with a water-insoluble film which has, for example, the following composition:

| | |
|---|---|
| Ethylcellulose | 40% |
| Triethyl citrate | 8% |
| Talc | 40% |
| Hydroxypropylcellulose | 12% |

The preparation is either sprayed on organically or subjected to aqueous processing using customary aqueous dispersions. Depending on the desired lag time, for example from 2 to 10 mg/cm² are applied. The lag time can thus be varied in a range from 1 hour up to 6 hours.

Unless specifically indicated otherwise in this application, all proportions and percentage data are always based on weight and, unless otherwise indicated, on the total weight of the appropriate unit, such as pellet, core, intermediate layer etc.

The following examples illustrate the invention without restricting it. Examples 2, 3, 7 and 8 are reference examples or comparative examples.

### EXAMPLE 1

### Production of Microtablets with Delayed Release

a: Production of the Tablet Cores:

The following compounds are introduced and mixed in a customary high-speed pharmaceutical mixer.

| | |
|---|---|
| Fluvastatin - Na. | 333 g |
| Lactose | 137 g |
| Microcrystalline cellulose | 400 g |
| Hydroxypropylmethylcellulose | 20 g |

The mixture is processed to granules by wet granulation with sufficient water. The granules are dried in a tray drying oven or in another suitable apparatus and then mixed with 100 g of croscarmellose (swelling agent), 5 g of magnesium stearate and 5 g of colloidal silica previously screened through a 250 μm sieve, and compressed to tablets. The tablets had a diameter of 2 mm and a mass of approx. 6 mg.

b: Subcoat

A mixture of

| | |
|---|---|
| Hydroxypropylcellulose | 135 g |
| Titanium dioxide | 18 g |
| Talc | 18 g |
| PEG 6000 | 9 g |
| Microcrystalline cellulose | 120 g |

is dissolved or dispersed in 1400 ml of water and sprayed onto the previously described produced tablets in customary pharmaceutical apparatuses.
c: Application of the Water-Insoluble Film

A mixture of ethylcellulose (e.g. Herkules Aqualon type N14) 125 g, triethyl citrate 12.5 g, talc 100 g and hydroxypropylcellulose Klucel EF (pore former) 25 g is dissolved or dispersed in ethanol. The preparation is sprayed in customary pharmaceutical equipment onto the tablets produced as above and provided with an intermediate layer. A layer with a layer thickness of approx. 5 mg/cm² results, indicated for the pure water-insoluble polymer ethylcellulose.

### EXAMPLES 2 TO 5

In the same manner as described above, tablets are produced with a varying thickness of water-insoluble film. The different layer thickness was adjusted by changing the amount applied.

| | | |
|---|---|---|
| Example 2 | Application of 25 g of | Layer thickness |
| | ethylcellulose | 1 mg/cm² |
| Example 3 | Application of 50 g of | Layer thickness |
| | ethylcellulose | 2 mg/cm² |
| Example 4 | Application of 75 g of | Layer thickness |
| | ethylcellulose | 3 mg/cm² |
| Example 5 | Application of 100 g of | Layer thickness |
| | ethylcellulose | 4 mg/cm² |

### EXAMPLE 6

a: Production of Tablet Cores:

Uncoated tablets are produced as described in example 1.

b: Application of Intermediate Film

The intermediate coating is applied by means of powder layering technology as follows. 25 g of croscarmellose are mixed with 100 g of microcrystalline cellulose, 100 g of corn starch and 1.5 g of colloidal silicon dioxide. A solution of 100 g of povidone (e.g. Kollidon K25 BASF) in 1000 g of water is prepared. The uncoated tablets are sprayed simultaneously with the powder mixture and the aqueous binder solution in a rotary processor equipped with a powder spray-in device. This results in rounding off of the band edges, and a substance which swells greatly (croscarmellose) is applied.

c: Application of the Water-Insoluble Film

The microtablets produced as above and provided with a water-soluble intermediate layer are coated as described in example 1 with a water-insoluble film, although application does not take place by spraying on an organic solution of the excipients; on the contrary, a purely aqueous dispersion is used.

| | |
|---|---|
| Ethylcellulose dispersion | 500 g* |
| (e.g. Aquacoat ECD - FMC) | |
| Triethyl citrate | 30 g |
| Talc | 75 g |
| Hydroxypropylcellulose | 15 g |
| Water | 730 g |

*stated as 30% strength dispersion

A layer thickness of 6 mg/cm² is applied (calculated as pure polymer ethylcellulose).

### EXAMPLES 7 TO 11

Tablets with a varying thickness of water-insoluble film are produced in the same manner as described in example 6.

| | | |
|---|---|---|
| Example 7 | Application of 25 g of | Layer thickness |
| | ethylcellulose | 1 mg/cm² |
| Example 8 | Application of 50 g of | Layer thickness |
| | ethylcellulose | 2 mg/cm² |
| Example 9 | Application of 75 g of | Layer thickness |
| | ethylcellulose | 3 mg/cm² |
| Example 10 | Application of 100 g of | Layer thickness |
| | ethylcellulose | 4 mg/cm² |
| Example 11 | Application of 125 g of | Layer thickness |
| | ethylcellulose | 5 mg/cm² |

### TEST EXAMPLE

The medicaments produced in the above examples were investigated for their release characteristics using the method already described. The results are listed in the table below (stated in each case as average of n=6 determinations). The exact release profiles of examples 1-5 are depicted in graph 1.

| | | Total amount | | Active |
|---|---|---|---|---|
| | | of active | | ingredient |
| | | ingredient | | released at |
| | | released | Duration of | the end of |
| | | at the end | accelerated | the |
| | | of the lag | release | accelerated |
| Example | Lag time [min] | time [%] | [min] | release |
| 2 | 51 | 0.5 | 22 | 102.2 |
| 3 | 115 | 0.4 | 29 | 99.8 |
| 4 | 195 | 0.4 | 35 | 103.1 |
| 5 | 270 | 0.4 | 40 | 101.5 |
| 1 | 345 | 0.5 | 42 | 98.0 |
| 7 | 15 | 2.5 | 10 | 99.5 |
| 8 | 45 | 3.6 | 12 | 101.2 |
| 9 | 120 | 1.8 | 15 | 102.6 |
| 10 | 160 | 6.2 | 21 | 98.3 |
| 11 | 210 | 4.5 | 35 | 100.2 |
| 6 | 260 | 2.8 | 42 | 101.6 |

1. A medicament containing at least one active ingredient which lowers the cholesterol level in the blood, comprising a means for releasing the active ingredient wherein the active ingredient is released with at least two different release rates, with a first release rate in a first period and with a second release rate, which is higher than the first release rate, in a subsequent second period, where the second period starts 2 to 12 hours after administration of the medicament, and where the means for releasing does not exclusively comprise an enteric coating.

2. The medicament as claimed in claim 1, wherein the active ingredient is an HMG-CoA reductase inhibitor and/or an active ingredient from the class of fibrates.

3. The medicament as claimed in claim 1, wherein the first period starts with administration of the medicament and lasts up to 10 hours.

4. The medicament as claimed in claim 1, wherein not more than 20% by weight of the active ingredient of the medicament is released within the first period.

5. The medicament as claimed in claim 1, wherein the first release rate is in the range from 0% active ingredient to 5% active ingredient and the second release rate is in the range from 6% to 100% active ingredient, in each case in a 10-minute interval.

6. The medicament as claimed in claim 1, wherein the first release rate is not equal to 0, and the second release rate is at least twice as high as the first release rate.

7. The medicament as claimed in claim 1, wherein the second period has a duration in the range from 15 minutes to 3 hours.

8. The medicament as claimed in claim 1, wherein the second period has a duration in the range from 3 to 6 hours.

9. The medicament as claimed in claim 1, wherein at least 50% by weight of the active ingredient of the medicament are released within the second period.

10. The medicament as claimed in claim 1, wherein the active ingredient used comprises one or more HMG-CoA reductase inhibitors selected from the group consisting of fluvastatin, simvastatin, atorvastatin, pravastatin, lovastatin, cerivastatin, nisvastatin, dolvastatin, bervastatin and rosuvastatin, enantiomers and enantiomer mixtures thereof, and pharmaceutically acceptable salts, hydrates and solvates thereof.

11. The medicament as claimed in claim 1, comprising a time-controlled system of the explosion type, in which an active ingredient-containing core is provided with one or more layers, where at least one of the layers represents a water-insoluble layer, the active ingredient-containing core comprises a swellable excipient, and the higher second release rate is brought about by explosive destruction of the water-insoluble layer.

12. The medicament as claimed in claim 11, wherein one or more intermediate layers, at least one of which comprises a swellable excipient, are applied between the active ingredient-containing core and the water-insoluble layer.

13. The medicament as claimed in claim 11, wherein the swellable excipient comprises a disintegrant.

14. The medicament as claimed in claim 11, wherein the water-insoluble film comprises ethylcellulose, cellulose acetate, polyvinyl acetate, acrylate or mixtures of these polymers, and optionally one or more customary excipients.

15. The medicament as claimed in claim 1, which comprises a system of two different types of particles, which are coated in such a way that:
the active ingredient is released slowly in a first period, then active ingredient is released faster in a second period following directly thereon, the active ingredient is released slowly in a third period following directly thereon, and the active ingredient is released faster in a fourth period following directly thereon.

16. The medicament as claimed in claim 1, which comprises a system of two different types of particles with two different active ingredients, which are coated in such a way that:
both active ingredients are released slowly in a first period, one active ingredient continues to be released slowly, but the second active ingredient is released faster, in a second period following directly thereon, the first active ingredient continues to be released slowly, while the second active ingredient has already been essentially completely released, in a third period following directly thereon, and the first active ingredient is released faster in a fourth period following directly thereon.

17. The medicament as claimed in claim 2, wherein the first period starts with administration of the medicament and lasts up to 10 hours.

18. The medicament as claimed in claim 2, wherein not more than 20% by weight of the active ingredient of the medicament is released within the first period.

19. The medicament as claimed in claim 2, wherein the first release rate is in the range from 0% active ingredient to 5% active ingredient and the second release rate is in the range from 6% to 100% active ingredient, in each case in a 10-minute interval.

20. The medicament as claimed in claim 2, wherein the first release rate is not equal to 0, and the second release rate is at least twice as high as the first release rate.

21. The medicament as claimed in claim 2, wherein the second period has a duration in the range from 15 minutes to 3 hours.

22. The medicament as claimed in claim 2, wherein the second period has a duration in the range from 3 to 6 hours.

23. The medicament as claimed in claim 2, wherein at least 50% by weight of the active ingredient of the medicament are released within the second period.

24. The medicament as claimed in claim 2, wherein the active ingredient used comprises one or more HMG-CoA reductase inhibitors selected from the group consisting of fluvastatin, simvastatin, atorvastatin, pravastatin, lovastatin, cerivastatin, nisvastatin, dolvastatin, bervastatin and rosuvastatin, enantiomers and enantiomer mixtures thereof, and pharmaceutically acceptable salts, hydrates and solvates thereof.

25. The medicament as claimed in claim 2, comprising a time-controlled system of the explosion type, in which an active ingredient-containing core is provided with one or more layers, where at least one of the layers represents a water-insoluble layer, the active ingredient-containing core comprises a swellable excipient, and the higher second release rate is brought about by explosive destruction of the water-insoluble layer.

26. The medicament as claimed in claim 25, wherein one or more intermediate layers, at least one of which comprises a swellable excipient, are applied between the active ingredient-containing core and the water-insoluble layer.

27. The medicament as claimed in claim 13, wherein the disintegrant is selected from the group consisting of crosslinked sodium carboxymethylstarch, crosslinked sodium carboxymethylcellulose, low-substituted sodium carboxymethylstarch, crosslinked PVP, low-substituted hydroxypropylcellulose, starch and a highly swellable ion exchange resin.

28. The medicament as claimed in claim 1, which comprises a system of two different types of pellets or two different types of microtablets, which are coated in such a way that:
the active ingredient is released slowly in a first period, then active ingredient is released faster in a second period following directly thereon, the active ingredient is released slowly in a third period following directly thereon, and the active ingredient is released faster in a fourth period following directly thereon.

29. The medicament as claimed in claim 1, which comprises a system of two different types of pellets or two different types of microtablets with two different active ingredients, which are coated in such a way that:
both active ingredients are released slowly in a first period, one active ingredient continues to be released slowly, but the second active ingredient is released faster, in a second period following directly thereon, the first active ingredient continues to be released slowly, while the second active ingredient has already been essentially completely released, in a third period following directly thereon, and the first active ingredient is released faster in a fourth period following directly thereon.

30. The medicament as claimed in claim 15, wherein the active ingredient is an HMG-CoA reductase inhibitor and/or an active ingredient from the class of fibrates.

31. The medicament as claimed in claim 16, wherein the active ingredient is an HMG-CoA reductase inhibitor and/or an active ingredient from the class of fibrates.

32. The medicament as claimed in claim 28, wherein the active ingredient is an HMG-CoA reductase inhibitor and/or an active ingredient from the class of fibrates.

33. The medicament as claimed in claim 29, wherein the active ingredient is an HMG-CoA reductase inhibitor and/or an active ingredient from the class of fibrates.
